(19) United States
(12) Patent Application Publication
(10) Pub. No.: US 2007/0173943 A1
(43) Pub. Date: Jul. 26, 2007

(54) Artificial nucleus pulposus and method of injecting same

(76) Inventors: Gary R. Dulak, Valencia, CA (US); Anil Bhalani, Mission Viejo, CA (US); Paul A. Molloy, St. Saviour (GB)

Correspondence Address: Gary R Dulak, 25860 Estaban Drive, Valencia, CA, 91355, US

(21) Appl. No.: 10/543,335

(22) PCT Filed: Jan. 16, 2004
(86) PCT No.: PCT/US04/01321

§ 371 Date: Jan. 2, 2007

Related U.S. Application Data
(60) Provisional application No. 60/441,038, filed on Jan. 17, 2003.

(51) Int. Cl. A61F 2/44

(52) U.S. Cl. 623/17.16

(57) ABSTRACT

The present invention relates to an artificial nucleus pulposus implant that is injected minimally invasively into the nucleus cavity of the annulus fibrosus to restore the normal anatomical and physiological function of the spine in the affected disc segment. In one aspect of the invention, a device is disclosed for delivering a phase changing biomaterial to a tissue site, the device comprising a dispenser including (i) a plunger having a proximal portion and a distal portion, an inlet end and an outlet end, (ii) a dispensing actuator attached to the proximal portion of the plunger, and (iii) a cartridge adapted to be inserted into the inlet end of the plunger for containing the phase changing biomaterial in a fluid state. The dispenser may be mechanically, pneumatically or hydraulically actuated. The dispenser may further comprise a nozzle attached to the cartridge for dispensing the biomaterial to the tissue site. In another aspect, the device may further comprise a tissue cavity access unit providing a conduit having an inlet end in fluid communication with the nozzle, and an outlet end adapted to deliver the biomaterial to the tissue site. The biomaterial may transition from the fluid state to a solid state after a set amount of time, a temperature change or an exposure to an external stimuli such as radiation, UV light or an electrical stimuli. The cartridge may be a dual-chambered cartridge for storing different fluid biomaterials in the two chambers. In another aspect of the invention, a process for producing the artificial nucleus pulposus implant in the nucleus cavity of the annulus fibrosus is disclosed, the process comprising the steps of (a) obtaining access to the nucleus cavity; (b) injecting the artificial nucleus pulposus into the nucleus cavity; and (c) permitting the biomaterial to transition from a fluid state to a solid state in-situ after a given condition.

This is a non-provisional application claiming the priority of provisional application Ser. No. 60/441,038, filed on Jan. 17, 2003, entitled “Artificial Nucleus Pulposus,” which is fully incorporated herein by reference.

### BACKGROUND OF THE INVENTION

1. Field of the Invention

The invention generally relates to artificial intervertebral disc nucleus and, more particularly, to an injectable artificial disc nucleus having the ability to restore the natural anatomical and physiological function of a degenerative disc.

2. Discussion of Related Art

Back pain is the number one reason for family doctor visits in the U.S., affecting more than 10 million people and is the single largest cause of healthcare expense in the country, amounting yearly to more than $50 billion in indirect and direct medical expenses. Drs. Rogers and Harrington pioneered the early work on which much of modern spinal surgery is still based. Since the 1940's a series of rod, hook and cage systems have evolved and since the 1980's “bone screws” have accompanied them. Pedicle screws became the new standard at this time due to high rates of fusion success. Although setbacks were experienced due to stress failures, better patient selection and a refinement of indications for use have seen the re-emergence of this technique. Threaded fusion cages arrived as an adjunct to this therapy in order to provide greater stability but have also been plagued by stress failures and high re-intervention rates.

Multiple new products have arrived in the last ten years and are making significant inroads. Interbody spinal cages, cervical plating systems, electrical and microwave stimulation for fusion and pain and more recently, artificial discs, prosthetic disc nuclei and bone growth factors are all evolving along parallel paths. Even in light of these surgical advances, there is still a large need for less invasive surgery.

Referring to FIG. 1, there is shown an intervertebral disc 10 contained between a superior vertebrae 34 and an inferior vertebrae 36. Between each vertebrae and intervertebral disc 10 lies vertebral endplates 42. The intervertebral disc 10, shown in FIG. 2, can be broken down into two basic components: an outer surrounding structure known as an anulus fibrosus 12 and an inner cushioning material called a nucleus pulposus 14.

Nucleus pulposus 14 is a gelatinous, slightly compressible, hydrophilic mass that is located in the center of the disc except in the lumbar segment, where it has a slightly posterior position. The anulus fibrosus 12 is a tough outer covering composed of fibrocartilage that contains the nucleus pulposus 14.

When the nucleus pulposus bulges from or leaks out of the ruptured annulus fibrosus 12, it is a condition known as a “herniated disc.” A herniated nucleus pulposus 22 and ruptured anulus fibrosus 24 are illustrated in FIG. 3. The herniated nucleus can cause excruciating pain for the patient because of the resultant pressure applied to branches of the local nerve network 26. If the herniation occurs in the lower lumbar spine, the sciatic nerve may be compressed. In such an instance, the patient will typically experience radicular pain in their lower extremities.

Typically, the initial onset of pain will be managed using conventional methods such as physical therapy, bed rest, chiropractic therapy, acupuncture, injection therapy or orthoses. If this “conservative management” does not alleviate the pain after several months of treatment and the imagining techniques show evidence of disc herniation, the physician may opt for surgical intervention.

Some patients and physicians opt to address the pain associated with this condition by completely removing the diseased disc and fusing the vertebrae above and below together, a procedure known as arthrodesis or spinal fusion. Not only is this procedure highly invasive, but also the objective of alleviating the pain is not always achieved and may be made worsened in some cases. In addition, by immobilizing a portion of the spine it has been found that there is an acceleration of disc degeneration in the discs above and below because of the altered biomechanics of the spine.

An alternative to spinal fusion is the use of intervertebral disc prosthesis. There are several devices disclosed in the prior art and several are in clinical trials that attempt to replace the natural intervertebral disc with an artificial disc. U.S. Pat. No. 3,867,728, to Stubstad et al., relates to a device which replaces the entire disc. This device is made by laminating vertical, horizontal or axial sheets of elastic polymer. U.S. Pat. No. 4,309,777, to Patil, relates to a prosthetic utilizing metal springs and cups. A spring implant comprising a rigid solid body having a porous coating on part of its surface is shown in Kenna's U.S. Pat. No. 4,714,469. U.S. Pat. No. 4,911,718, to Lee et al., relates to an elastomeric disc spacer comprising a nucleus, an anulus and a plurality of end-plates, each of which is formed from different materials.

The primary disadvantage of the invention of Stubstad et al., Patil, Kenna and Lee et al., is the use of their prosthesis requires complete replacement of the natural disc which involves numerous surgical difficulties and significant trauma to the surrounding tissue. Secondly, the intervertebral disc is a complex joint, anatomically and functionally, comprising the aforementioned three different structures, each of which has its own unique structural characteristics. Designing and fabricating such a complicated prosthesis from acceptable materials, which will mimic the function of the natural disc, is very difficult. A further problem is the difficulty of preventing the prosthesis from dislodging.

A collapsible plastic bladder-like prosthetic of nucleus pulposus is disclosed by Froning in U.S. Pat. No. 3,875,595. An intervertebral disc prosthetic comprising of a pair of rigid plugs to replace the degenerated disc is referred by Kuntz, U.S. Pat. No. 4,349,921. U.S. Pat. Nos. 4,772,287 and 4,904,260, to Ray et al., teach the use of a pair of pre-molded, cylindrical prosthetic intervertebral disc capsules enclosed within a flexible, inelastic, woven polyethylene jacket.

These problems are not solved by Kuntz, who uses elastic rubber plugs, or by Froning and Ray et al., who use bladders, or capsules, respectively, which are filled with a fluid or thixotropic gel. According to the Ray and Froning patents, liquid was used to fill the capsules and bladders, respectively, thereby requiring that their membranes be completely sealed to prevent fluid leakage. As a consequence, those devices cannot completely restore the function of the nucleus which allows body fluid to diffuse in and out during cyclic loading thereby providing the nutrients the disc needs.

Even for prosthesis that are only intended for replacing the nucleus, a major obstacle has been to find a material which is similar to the natural nucleus and is able to restore the normal function of the nucleus. Hydrophobic elastomers and thermoplastic polymers are not desirable for use in the prosthetic nuclei due to their significant inherent differences from the natural nucleus, e.g., lack of hydrophilicity in the elastomers and lack of flexibility in the thermoplastics.

Ross and Guagliano, in U.S. Pat. Nos. 6,183,518, 6,206,921 and 6,436,143, describe the implantation of a latex material into the nucleus cavity. The biocompatibility, injection temperature, and hydrophobic nature of the material are major disadvantages of the Ross et al. inventions.

The Newcleus, manufactured by Sulzer-SpineTech, currently in development, utilizes an elongated elastic memory-coiling spiral made of polycarbonate urethane. It is inserted through a postero-lateral annulotomy after discetomy, and then is designed to form spiral coils within the annulus to fill the nuclear cavity.

Bao et al., in U.S. Pat. Nos. 5,047,055 and 5,192,326, describe artificial nuclei comprising hydrogels in the form of large pieces shaped to conform to the shape of the disc cavity or beads within a porous envelope, respectively. Bao et al., in U.S. Pat. No. 6,280,475, describes the use of pre-molded xerogel rods that are used to replace the natural nucleus. U.S. Pat. No. 6,264,695, to Stoy, relates to anisotropically swellable, biomimetic xerogel plastic that is used as a prosthetic nucleus. One of the major disadvantages in these inventions is the requirement for the hydrogel article to be pre-molded and implanted into the nucleus. Bao et al. and Stoy describe a xerogel that is implanted in a dehydrated state. The implantation of a pre-molded article still requires a larger incision in the surrounding tissue and the unnecessary need for further trauma. The numerous advantages offered by a hydrogel material in this application and described by Bao et al., Stoy, and Ray et al. are highlighted below.

Hydrogels have been used in biomedical applications, such as contact lenses and wound dressings. Among the advantages of hydrogels is that they are more biocompatible than hydrophobic elastomers and metals. This biocompatibility is largely due to the unique characteristics of hydrogels in that they are soft and contain water like the surrounding tissues and have relatively low frictional coefficients with respect to the surrounding tissues. The biocompatibility of hydrogels results in prosthetic nuclei, which are more easily tolerated in the body. Furthermore, hydrophobic elastomeric and metallic gels will not permit diffusion of aqueous compositions, and the solutes, there through.

An additional advantage of some hydrogels is their good mechanical strength, which permits them to withstand the load on the disc, to restore the normal space between the vertebral bodies, and to assist in the healing of the defective annuli. Other advantages of the hydrogels are their excellent viscoelastic properties and shape memory. Hydrogels contain a large amount of water, which acts as a plasticizer. Part of the water is available as free water, which has more freedom to leave the hydrogel when the hydrogel is partially dehydrated under mechanical pressure. This characteristic of the hydrogels enables them to creep, in the same way as the natural nucleus, under compression, and to withstand cyclic loading for long periods without any significant degradation or loss of their elasticity.

Another advantage of hydrogels is their permeability to water and water-soluble substances, such as nutrients, metabolites and the like. It is known that body fluid diffusion, under cyclic loading, is the major source of nutrients to the natural disc since the disc itself is relatively avasular. If the route of this nutrient diffusion is blocked, e.g., by a water-impermeable nucleus, further deterioration of the disc will ensue.

Another alternative treatment option available to the patient is a microdisectomy. A microdisectomy is a minimally invasive procedure to remove the herniated nucleus pulposus material and relieve the associated pressure on the local nerve network. This procedure provides the patient with short-term pain relief in a majority of the cases, however, it introduces some long-term complications.

Referring to FIG. 4, there is shown a side view of the anulus fibrosus 12 located between the superior vertebrae 34 and inferior vertebrae 36. Within the inner layers of the anulus 12, there is a crisscross network of coarse collagen fiber bundles 32 attached to the vertebrae above and below. The collagen fibers 32 are designed to support high bending movements, torsional loads and radial forces applied by the constrained nucleus. The fibers 32 are about 25 nm to about 40 nm in diameter and have a greater tensile strength than any synthetic fiber. Although strong in tension, collagen fibers offer little resistance in compression.

FIG. 5 is a simple illustration of the force transfer mechanism within an intervertebral disc. When a compressive load 44 is applied in the axial direction from the vertebrae above, the inherent hydraulic properties of the nucleus transfers the load radially 46 to the surrounding anulus. When the load transfer occurs, the anulus 12 begins to expand laterally and is further restricted by the circumferential tension in the network of fibers in the anulus. Stated another way, the anulus 12 is designed to bear a majority of the spinal load in the radial direction and not in the axial direction.

After a microdisectomy procedure, the anulus is absent of the nucleus and thus must bear the entire spinal load in the axial direction. For the same given axial load, the compressive stress (load per unit area) will more than double due to the decrease in surface area bearing the load. The alteration in the biomechanics of the spine due to the absence of a nucleus cushion decreases the life of the anulus because it is not being utilized in the capacity for which it was designed. The resultant alteration in stress sharing may lead to accelerated disc degeneration.

As such, there is a significant gap between the available conservative therapies for the treatment of degenerative disc and the highly invasive surgical procedures for repair. The disabling pain that accompanies the disorder further fuels the race to develop a better treatment option. A replacement, augmentative material placed into the intervertebral disc minimally invasively and functioning as closely as possible to the original nucleus pulposus would be an ideal method for addressing disc herniation. While development efforts may be underway to develop such a material, none is currently available. It is deemed that the hydro-polymer artificial nucleus pulposus described as part of the present invention together with methods of delivering the material to the nucleus of the disc represent a significant advance compared to existing prior art discussing prosthetic nucleus replacement.

### SUMMARY OF THE INVENTION

The present invention relates to an artificial nucleus pulposus implant that is injected minimally invasively into the nucleus cavity of the anulus fibrosus to restore the normal anatomical and physiological function of the spine in the affected disc segment.

In one aspect of the invention, it is directed to a device for delivering a phase changing biomaterial to a tissue site, the device comprising a dispenser that includes (i) a plunger having a proximal portion and a distal portion, an inlet end and an outlet end, (ii) a dispensing actuator attached to the proximal portion of the plunger, and (iii) a cartridge adapted to be inserted into the inlet end of the plunger for containing the phase changing biomaterial in a fluid state. The dispenser may be mechanically actuated, pneumatically actuated, or hydraulically actuated. The dispenser may further comprise a nozzle attached to the cartridge for dispensing the biomaterial to the tissue site. In another aspect of the invention, the device may further comprise a tissue cavity access unit providing a conduit having an inlet end in fluid communication with the nozzle, and an outlet end adapted to deliver the biomaterial to the tissue site. It is appreciated that the biomaterial may transition from the fluid state to a solid state after a set amount of time, a temperature change, or an exposure to an external stimuli such as radiation, UV light, or an electrical stimuli.

The cartridge may be a dual-chambered cartridge for storing a first fluid biomaterial in a first chamber and a second fluid biomaterial in a second chamber. In one aspect of the invention, the first fluid biomaterial may include hydrophilic poly(aldehyde) and the second fluid biomaterial may include at least one of poly(amide), poly(amine) and poly(alcohol). In another aspect of the invention, the first fluid biomaterial may include a poly(n-vinyl lactam) component and the second fluid biomaterial may include a chitosan component. In yet another aspect of the invention, the tissue cavity access unit comprises an entry needle, an access cannula, and an obturator. The cannula and obturator are adapted to dilate tissue of the annulus fibrosus, and are comprised of a thermopolymer such as PTFE, polyurethane, polyethylene, Pebax, polyester, polycarbonate, nylon, or delrin, or a metal such as stainless steel or Nitinol. The biomaterial of the invention may comprise a plurality of biomaterial components including a mixture of water and polyethyleneoxide/polypropyleneoxide (PEO-PPO) non-ionic block copolymer. The biomaterial components may further comprise at least one of polyethyleneoxide (PEO) homopolymer, polypropyleneoxide (PPO) homopolymer, and other hydrophilic compounds including surfactants, alcohols, acids, salts, amines and mixtures thereof.

Another aspect of the invention is directed to a process for producing an artificial nucleus pulposus implant in the nucleus cavity of the annulus fibrosus of a diseased disc to improve the natural anatomical and physiological function of the disc, the process comprising the steps of (a) obtaining access to the nucleus cavity; (b) injecting the artificial nucleus pulposus into the nucleus cavity, the artificial nucleus pulposus comprising a phase changing biomaterial; and (c) permitting the biomaterial to transition from a fluid state to a solid state in-situ after a given condition. The process of the invention may further comprise the step of removing the natural nucleus pulposus from the nucleus cavity before the step of injecting the artificial nucleus pulposus in the nucleus cavity. It is appreciated that during the process of the invention, the biomaterial may transition from the fluid state to the solid state after a set amount of time, a temperature change, or an exposure to an external stimuli such as radiation, UV light, or an electrical stimuli. The natural nucleus pulposus removing step may include one of irrigation, aspiration, chemonucleolysis, and grasping. It is preferable that the biomaterial components have a viscosity of less than about 5,000 cps in the fluid state and a viscosity of greater than about 100,000 cps in the solid state.

In another aspect of the invention, the artificial nucleus pulposus injecting step further comprises the step of mixing the biomaterial components, which may include a first fluid biomaterial and a second fluid biomaterial. The first fluid biomaterial may include hydrophilic poly(aldehyde) and the second fluid biomaterial may include at least one of poly(amide), poly(amine) and poly(alcohol). In another aspect, the first fluid biomaterial may include a poly (n-vinyl lactam) component and the second fluid biomaterial may include a chitosan component. Similarly to the device of the invention, the biomaterial components may include a mixture of water and polyethyleneoxide/polypropyleneoxide (PEO-PPO) non-ionic block copolymer, or the biomaterial components may further comprise at least one of polyethyleneoxide (PEO) homopolymer, polypropyleneoxide (PPO) homopolymer, and other hydrophilic compounds including surfactants, alcohols, acids, salts, amines and mixtures thereof. In yet another aspect of the invention, the process of the invention may be performed using endoscopic surgical instrumentation. The process of the invention may also be performed with the assistance of fluoroscopy or other imaging or resolution enhancing instrument.

In another aspect of the invention, a process for producing an artificial nucleus pulposus implant in the nucleus cavity of the annulus fibrosus of a diseased disc is disclosed to improve the natural anatomical and physiological function of the disc, the process comprising the steps of (a) obtaining access to the nucleus cavity; (b) inserting a scaffold in the nucleus cavity; and (c) injecting the artificial nucleus pulposus in the nucleus cavity, the artificial nucleus pulposus including a phase changing biomaterial. It is preferable that the process of the invention further comprises the step of permitting the biomaterial to transition from a fluid state to a solid state in-situ after a given condition. The process may further comprise the step of removing the natural nucleus pulposus from the nucleus cavity before the step of injecting the artificial nucleus pulposus in the nucleus cavity. The scaffold may be made from preformed, extruded metal or high durometer plastic such as polyurethane, polyethylene, silicone and PTFE. In another aspect, the scaffold is made of an injectable foam that solidifies in-situ.

In yet another aspect of the invention, a process for repairing a diseased disc to restore the natural anatomical and physiological function of the disc is disclosed, the process comprising the steps of (a) providing an apparatus for delivering a phase changing biomaterial to the disc in a minimally invasive manner; (b) providing the phase changing biomaterial to be injected to the disc; and (c) permitting the biomaterial to transition from a fluid state to a solid state in-situ after a given condition. During the process of the invention, the phase changing biomaterial includes a plurality of biomaterial components adapted to be mixed at the time of use to initiate cure. The process may further comprise the step of mixing the biomaterial components to initiate cure and delivering the mixed biomaterial to the disc in the fluid state. It is appreciated that minimally invasive techniques such as irrigation, aspiration, chemonucleolysis and grasping may be used to remove the damaged or diseased nucleus pulposus from the disc. As such, in all of the embodiments of the invention, the artificial nucleus pulposus will as closely as possible restore normal anatomical and physiological function of the affected disc.

“First do no harm” is a fundamental of the Hippocratic Oath. To leave as much as possible of the normal anatomy and physiology of the patient intact and unharmed is a central tenet of any intervention. Spinal fusion, disc laminectomy, disc laminotomy and disc replacement are invasive and injurious surgical techniques associated with a wide variation of desired outcomes. Procedures that are less invasive than spinal fusion, such as the invention disclosed by Ray et al., still envisage the removal of spinal structures for the purpose of access to the operative site. Specifically, Ray et al. teaches the need to remove the lamina (laminectomy) in order insert a prosthetic spinal disc nucleus. One advantage of the artificial nucleus pulposus system of the invention represents a treatment modality that is not only significantly less traumatic than current techniques, but it is also a method that is designed to leave undisturbed as much of the normal and useful anatomy of the patient as possible. In relying upon the salvage of the anulus fibrosus the method envisages an interruption of the expected disease process by approaching normal restoration of disc function. By providing an analogue for the natural nucleus pulposus and by negating the irreversible removal or modification of the anulus the method is augmentative and restorative of remaining natural tissue and complimentary to the physical dynamics of the spine.

Delivering the artificial nucleus pulposus will drastically decrease the invasiveness of repairing a herniated disc surgically. The proposed repair option may be expected to be less painful, of shorter duration and related to a lower incidence of associated morbidities than the prior art and therefore more favorable to the patient. The aforesaid clinical advantages may also be reasonably expected to result in lower average operating procedure costs and lower average hospital costs attributable to an expected reduction in the length of stay at the care facility. These savings and advantages are expected to translate overall to a decrease in the social burden associated with the incidence of chronic back pain.

An additional advantage of this invention is no requirement to determine the size of the implant needed. Given that the artificial disc nucleus is in fluid form when delivered, it will fill a wide array of cavity sizes. This is beneficial from a hospital inventory perspective where only one product will need to be stocked. The physician will not have to be concerned whether the correct size is in stock and will be assured of the “best fit” for a particular patient after each delivery, something that cannot be said about preformed devices, which, by definition, are not particular to an individual.

Another advantage of this invention is that the artificial nucleus pulposus will completely fill the nucleus cavity restoring the desired biomechanics of the spine. Complete fill of the nucleus cavity will allow the axial forces experienced by the intervertebral disc to be accurately transferred into a radial force that is resisted by the anulus fibrosus, as the anulus fibrosus was designed for.

Another advantage of this invention is the patient's vertebra will not need to be “jacked-up”, a technique involving the creation of additional intervertebral space by means of a mechanical lever. Since the amount of material delivered to the nucleus cavity is limited to completely fill the available space within the annulus, no such artificial heightening is required

Another advantage of this invention is the reduced possibility of re-herniation of the artificial nucleus pulposus relative to the prior art because the substantially greater ratio of the implant size to the annulus fibrosus insertion port. All of the prior art discusses the implantation of a pre-molded prosthetic that requires the container, anulus fibrosus, to be incised by approximately the same size as the implant. This invention only requires the container to be incised by a fraction of the size of the nucleus cavity because it can be delivered in fluid form thus reducing the possibility of re-herniation once the artificial nucleus pulposus has molded in-situ.

These and other features and advantages of the invention will become more apparent from the following description of preferred embodiments in reference to the associated drawings. It is to be understood that the drawings are to be used for the purposes of illustration only and not as a limitation on the scope of the invention.

### DESCRIPTION OF THE DRAWINGS

FIG. 1 is a sagittal view of the intervertebral motion segment;

FIG. 2 is a cross-sectional, elevational view of FIG. 1 showing the anatomy of the intervertebral disc;

FIG. 3 is a cross-sectional, elevational view of FIG. 1 illustrating a herniated nucleus compressing a nerve;

FIG. 4 is a side view of the anulus fibrosus highlighting the criss-cross network of collagen fibers;

FIG. 5 is a sectional view of FIG. 4 showing the distribution of a spinal load;

FIG. 6 shows a perspective view of the mechanically actuated dispenser;

FIG. 7 shows a perspective view of the dual-chambered cartridge;

FIG. 8 shows a perspective view of the static mixing nozzle;

FIG. 9 shows a perspective view of the entry needle, access cannula, and obturator used to access the nucleus cavity;

FIG. 10 is a cross-sectional, elevational view of FIG. 1 illustrating access into the nucleus cavity;

FIG. 11 is a cross-sectional, elevational view of FIG. 1 showing a conduit into the nucleus cavity via the access cannula;

FIG. 12 is a cross-sectional, elevational view of FIG. 1 exhibiting the removal of the natural nucleus from the nucleus cavity;

FIG. 13 is a cross-sectional, elevational view of FIG. 1 depicting the filling of the nucleus cavity with an artificial nucleus pulposus in a fluid state;

FIG. 14 is a cross-sectional, elevational view of FIG. 1 showing a completely filled nucleus cavity with an artificial nucleus pulposus in a solid state; and

FIGS. 15-17 illustrate the steps of an alternative embodiment of the artificial nucleus pulposus, using a metal scaffold.

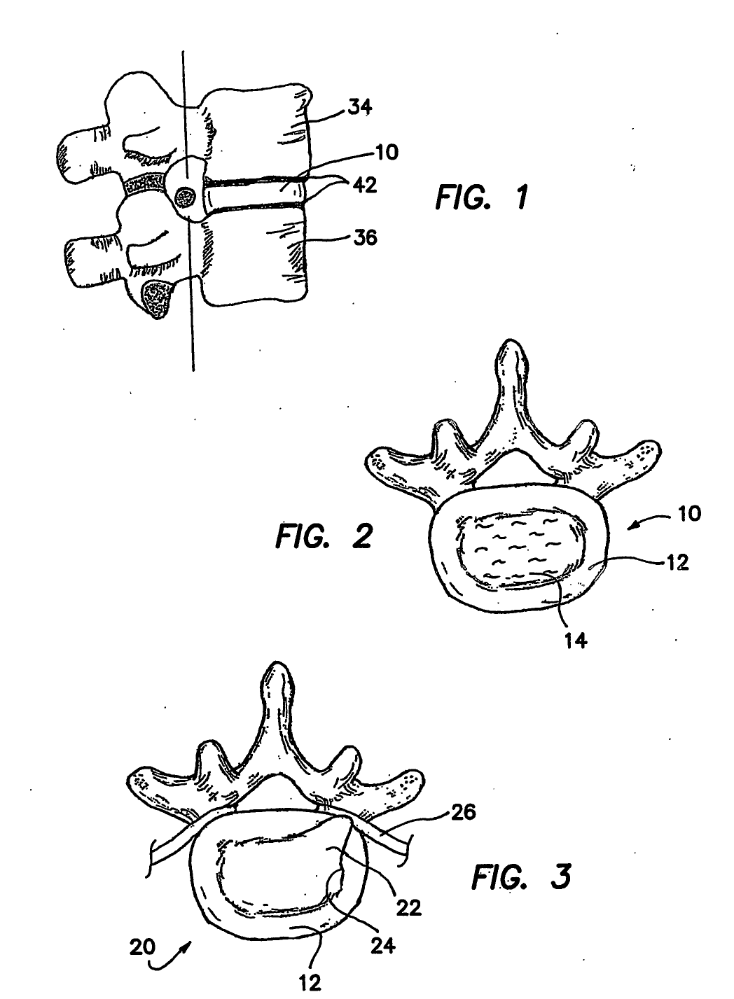

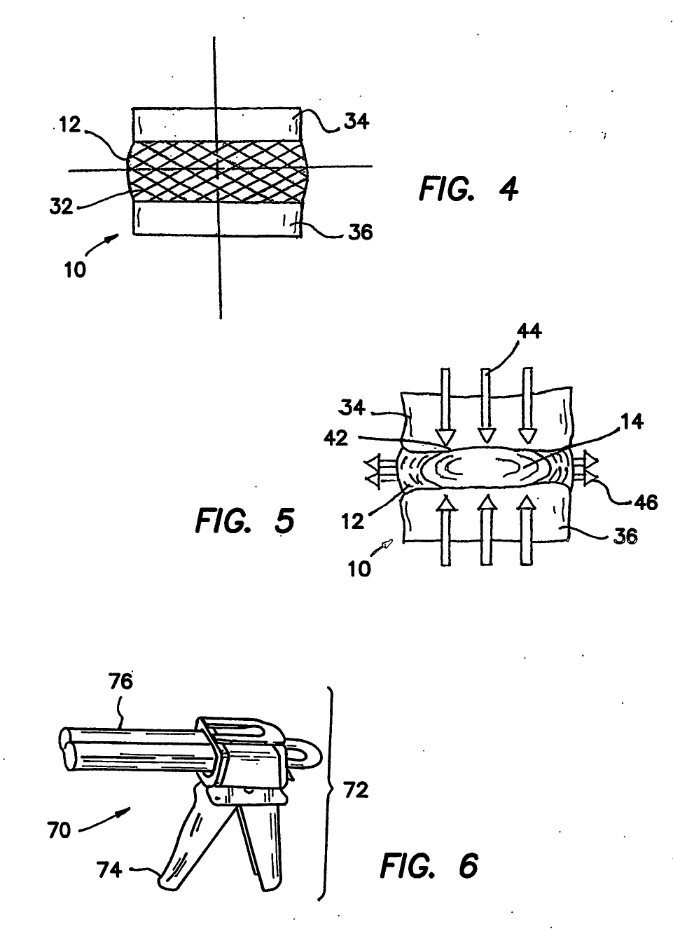

### DESCRIPTION OF PREFERRED EMBODIMENT AND BEST MODE OF THE INVENTION

The following is a list of reference numerals as used in the drawings of the present invention:

### LIST OF REFERENCE NUMERALS

10 Intervertebral Disc
12 Anulus Fibrosus
14 Nucleus Pulposus
20 Herniated Disc
22 Herniate nucleus pulposus
24 Anulus tear/fissure
26 Compressed nerve
32 Collagen fiber
34 Superior vertebrae
36 Inferior vertebrae
42 Vertebral end-plate
44 Compressive load
46 Radial force
51 Nucleus cavity
52 Entry needle
53 Obturator
54 Access Cannula
55 Obturator/cannula assembly
61 Suction/aspirating catheter
70 Mechanically actuated dispenser
72 Body of dispenser
74 Trigger of dispenser
76 Plunger of dispenser
80 Dual-chambered cartridge
81 Chamber A of cartridge
82 Part A of artificial nucleus pulposus
83 Chamber B of cartridge
84 Part B of artificial nucleus pulposus
86 Cartridge tip
90 Static mixing nozzle
91 Distal end of static mixing nozzle
92 Base of static mixing nozzle
94 Mixing fins
102 Artificial nucleus pulposus (Fluid)
104 Artificial nucleus pulposus (Solid)
104 Artificial nucleus pulposus (Solid)
152 Scaffold Article
154 Scaffold (Gathered article)

The device according to this invention is designed to replicate the structure and material properties of the natural nucleus pulposus to the extent needed to restore all the essential functions. The preferred spinal nucleus implant according to the present invention has properties closely mimicking the essential properties of natural nucleus pulposus, such as affinity for water absorption, spinal load transfer, fluid transport of nutrients and excretions, and cushion for spinal loads.

The spinal nucleus implant according to the present invention also has the following differences from natural nucleus pulposus: synthetic material that has two-phases (fluid and solid), one-piece mold form that has internal bonds, higher durometer, visco-elastic, and radiopaque.

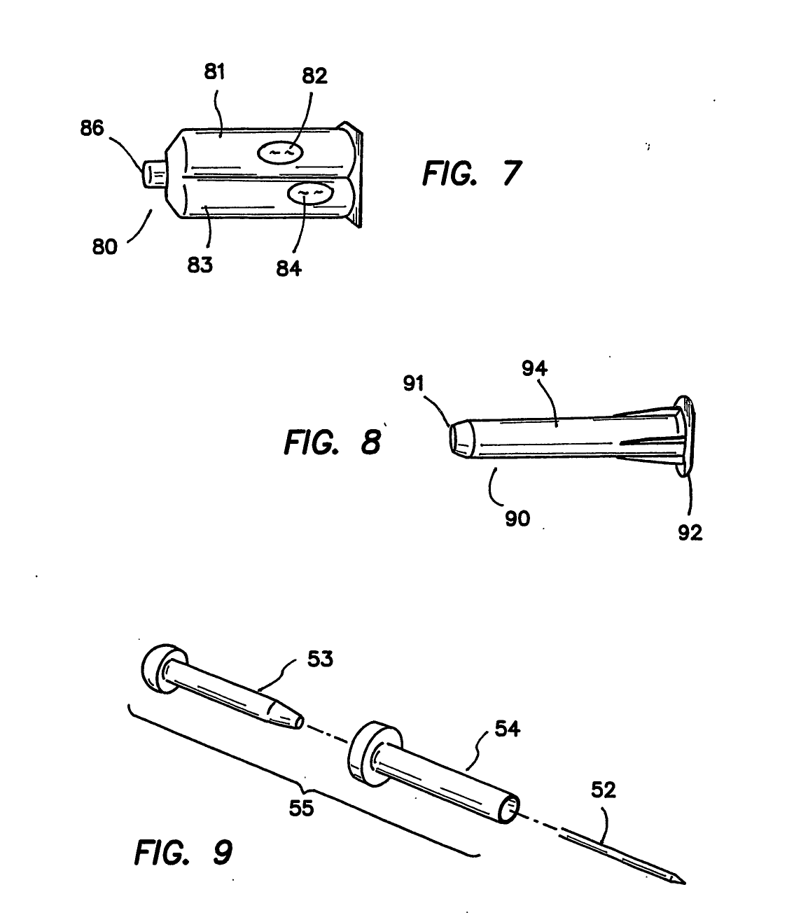

Referring to FIGS. 6-9, the preferred embodiment of the delivery device for the artificial nucleus pulposus comprises of three basic components: a mechanically actuated dispenser 70 as illustrated in FIG. 6, a dual-chambered cartridge 80 as illustrated in FIG. 7, and a static mixing nozzle 90 as illustrated in FIG. 8. Mechanically actuated dispenser 70 further comprises a body 72, a trigger 74 and a plunger 76. When the trigger 74 is squeezed against the body 72, the plunger 76 is advanced forward.

FIG. 7 depicts the dual-chambered cartridge 80 having two separate chambers to store two fluid components of the un-reacted implant material. Chamber A 81 contains a first fluid component, referred to as Part A 82, and chamber B 83 contains a second fluid component, herein referred to as Part B 84. As the plunger 76 is advanced, the two fluid components contained within each of the chambers are expelled from the respective chambers and extruded through a cartridge tip 86.

FIG. 8 depicts a static mixing nozzle 90 with a base 92 that is attached to the cartridge tip 86 with a “bayonet” type of attachment. As the fluid components 82 and 84 are pressed through the static mixing nozzle 90, small amounts of Part A 82 and Part B 84 are exchanged within the static mixing nozzle 90 and mixed as they encounter numerous mixing fins 94 that promote the mixing of Part A 82 and Part B 84. At a distal end 91 of the static mixing nozzle 90, a homogenous artificial nucleus pulposus 102 (see, e.g., FIG. 12) is extruded.

Referring to FIG. 9, there are shown accessories to access the nucleus cavity 51. The components of this access assembly include: an entry needle 52, an obturator 53, and an access cannula 54. Entry needle 52 is a small diameter tool with an outer diameter of about 0.010″ to about 0.100″ that is used to access the nucleus cavity 51 and to provide a “rail” to facilitate the passage of other instrumentation into the nucleus cavity 51. A larger diameter cannula/obturator assembly 55 having an outer diameter of about 0.050″ to about 0.400″ and an inner diameter slightly larger then the entry needle is used to dilate the tissue of the anulus 12. The distal end of the cannula/obturator assembly 55 has a tapered profile and a low coefficient of friction. The cannula/obturator is made of a material such as PTFE, polyurethane, polyethylene, Pebax, polyester, polycarbonate, nylon, or delrin, or a metal such as stainless steel or nitinol, or other material that has a low coefficient of friction to allow for gradual dilation of the tissue. It is also known that a polymer or metal substrate can be coated with a “slick” coating such as a hydrophilic, paralene or PTFE coating to reduce the coefficient of friction of the substrate's surface. A PTFE material is the preferred material of this invention.

In one preferred embodiment of the artificial nucleus pulposus, chamber A 81 contains a hydrophilic poly(aldehyde), Part A 82, and chamber B 83 contains a poly(amide), poly(amine) or poly(alcohol) and mixtures thereof, Part B 84. Chamber A 81 and chamber B 83 are the same volume so as to have a 1:1 mixture of the components when they are pushed through the static mixing nozzle 90. The homogenous artificial nucleus pulposus 102 extruded from the distal end 91 of the static mixing nozzle 90 creating a fluid hydrogel. The possible compositions of the polymer components, mentioned above, used to create the hydro-polymer are described in greater detail by Eknoian in U.S. Pat. No. 6,365,664. It has been speculated that a covalent cross-linking dispersed through an interconnection network of ionic bonds in Part B occurs to form a solid, non-reversible gel.

In another embodiment of the invention of the artificial nucleus pulposus, chamber A 81 contains a poly(n-vinyl lactam) component, Part A 82, and chamber B 83 contains a chitosan component, Part B 84. Chamber A 81 and chamber B 83 have the same volume so as to have a 1:1 mixture of the components when they are pushed through the static mixing nozzle 90. The homogenous artificial nucleus pulposus 102 extruded from the distal end 91 of the static mixing nozzle 90 creates a fluid hydrogel. One type of these gel systems is thoroughly described by Lorenz et al. in U.S. Pat. No. 6,379,702. Depending on the cure time of the material, which is determined by the ratio of polymer components, a covalent cross-linking dispersed through an interconnection network of ionic bonds in Part B occurs to form a solid, reversible gel.

And yet in another embodiment of the invention is a temperature-responsive, single-part, two-phase gel system that transitions from a fluid to a solid state between about 70° F. and about 120° F.; and more preferably between about 85° F. and about 100° F. In other aspects of the invention, the biomaterial transitions from the fluid state to the solid state when exposed to UV light or to an electrical stimulation. A preferred gel composition includes a mixture of water and polyethyleneoxide/polypropyleneoxide (PEO-PPO) non-ionic block copolymer, which preferably contains additives, such as polyethyleneoxide (PEO) homopolymer and/or polypropyleneoxide (PPO) homopolymer, and other hydrophilic compounds such as surfactants, alcohols, acids, salts, amines and the like, or mixtures of additives thereof. By varying the concentration of a homopolymer or other additive in the base mixture/PEO-PPO block copolymer in water, the transition temperatures and the firmness of the gel can be adjusted as desired. This embodiment is a single-component system and therefore does not require the mixing of two components as mentioned in the previous embodiments of the artificial nucleus pulposus implant. Therefore, a dispenser for this gel system (not shown) is similar to the mechanically actuated dispenser 70 but only has a single plunger. In addition, this embodiment does not require the use of the static mixer 90.

When accessing the nucleus cavity Si, it is important to consider the surgical approach. It is well known that the nucleus cavity can be accessed using an “open technique.” This access technique requires the muscles to be dissected, tendons attachments to be severed, a portion of the spine to be removed (laminectomy), and the annulus fibrosus to be incised. The artificial nucleus pulposus of the invention is delivered in a fluid state via a cannula/catheter and therefore there is no need to use the open technique described above.

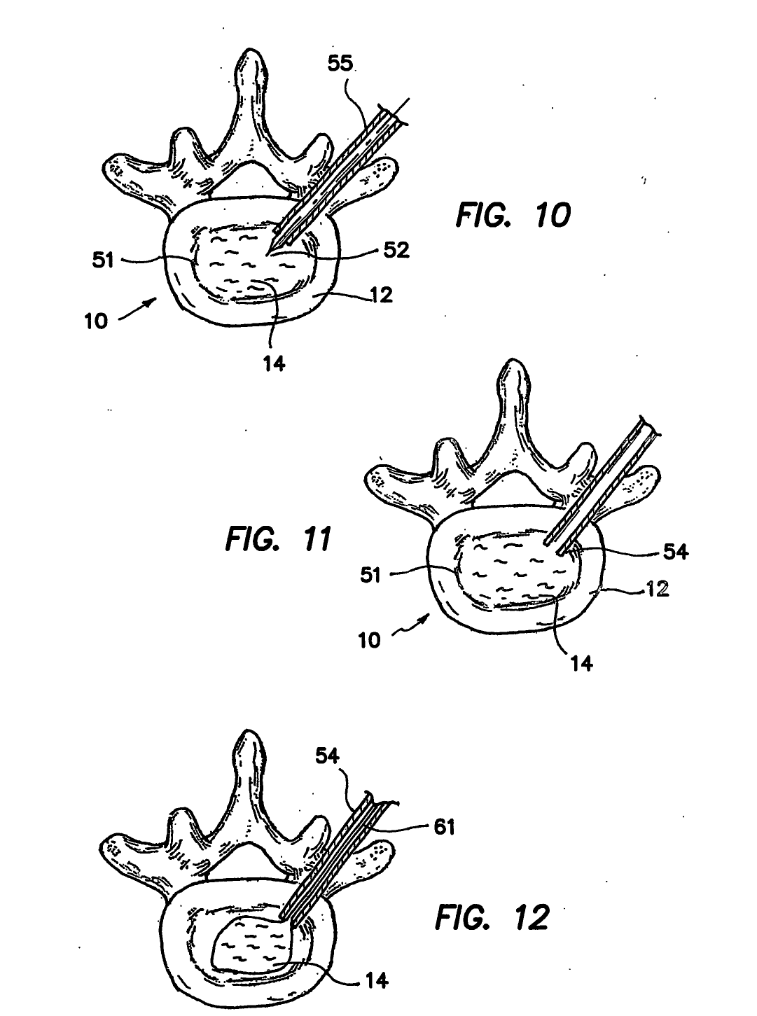

FIG. 10 details a preferred access technique, referred to as “tissue dilation”. The entry needle 52 is inserted through the anulus 12 and into the nucleus cavity 51. Once the entry needle 52 has been placed, the cannula/obturator assembly 55 is advanced co-axially over the entry needle 52 and inserted through the wall of the anulus fibrosus 12, gradually dilating the fibrosus cartilage as the assembly is advanced into the nucleus cavity 51.

After the assembly 55 has been located, the obturator 53 is removed to leave the access cannula 54 in place. Now, in effect, the surgeon has a clear conduit into the nucleus cavity 51 that effectively retracts the surrounding tissue with little trauma. FIG. 11 shows the obturator 53 removed and the access cannula 54 left in place.

If desired, a hole through and in the anulus 12 to access the nucleus cavity 51 can be incised to create a similar conduit. Even though this is not the most preferred access technique due to greater trauma to the anulus 12, it is another access technique available to the surgeon. Accessing the cavity using a “tissue-dilation” technique rather than an “apple-coring” technique will impart less trauma to the anulus 12 and provide the anulus with a greater opportunity to heal.

Once access to the nucleus cavity 51 has been obtained, the surgeon will remove the natural nucleus 14 using various techniques. Some techniques available to the surgeon are irrigation/aspiration, chemonucleolysis and metal graspers. FIG. 12 illustrates the removal of the natural nucleus 14 from the nucleus cavity 51 using a suction/aspirating catheter 61 located through the access cannula 54. After the partial or full removal of the natural nucleus 14 has been completed, the nucleus cavity 51 is prepared for the implantation/injection of the artificial nucleus pulposus.

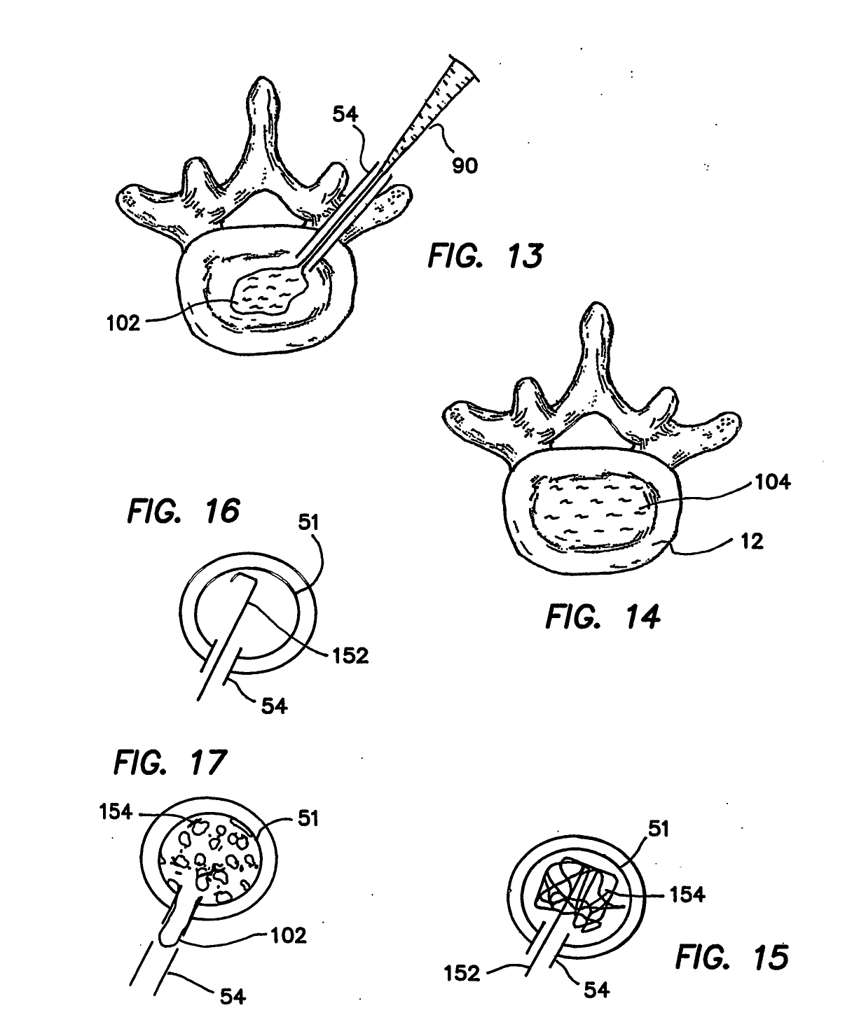

FIG. 13 illustrates the fluid, homogeneous artificial nucleus pulposus 102 injected directly into the nucleus cavity 51, from which the natural nucleus 14 had been excised. After a set amount of time or temperature change, the artificial nucleus pulposus transitions from a fluid state 102 to a solid state 104 as illustrated in FIG. 14, at which point the solid artificial nucleus pulposus 104 is constrained tightly therein by the annulus 12 and end plates (not shown). In the fluid state, prior to a cross-linking of the materials, the gel has a viscosity of less than about 5,000 cps. In the solid state and after cross-linking, the gel has a viscosity of greater than about 100,000 cps.

FIGS. 15-17 show an alternative embodiment of the artificial nucleus pulposus, which includes the addition of a metal scaffold. FIG. 15 illustrates the initial feeding of a preformed, extruded scaffold article 152. It is preferred that the scaffold material is a metal, however, a higher durometer plastic such as polyurethane, polyethylene, silicone, or PTFE could be used. FIG. 16 shows the scaffold article 154 gathering in the nucleus cavity when it is continuously inserted through the access cannula 54. FIG. 17 shows the artificial nucleus pulposus 102 injected over the scaffold 154 located within the nucleus cavity 51.

It will be understood that many other modifications can be made to the various disclosed embodiments without departing from the spirit and scope of the invention. For these reasons, the above description should not be construed as limiting the invention, but should be interpreted as merely exemplary of preferred embodiments.

1. A device for delivering a phase changing biomaterial to a tissue site, comprising:
(a) a dispenser (70) comprising:
(i) a plunger (76) having a proximal portion and a distal portion, an inlet end and an outlet end,
(ii) a dispensing actuator (74) attached to the proximal portion of the plunger (76), and
(iii) a cartridge (80) adapted to be inserted into the inlet end of the plunger (76) for containing the phase changing biomaterial in a fluid state.

2. The device of claim 1, wherein the dispenser (70) is mechanically actuated, pneumatically actuated, or hydraulically actuated.

3. The device of claim 1, wherein the dispenser (70) further comprises a nozzle (90) attached to the cartridge (80) for dispensing the biomaterial to the tissue site.

4. The device of claim 3, further comprising a tissue cavity access unit providing a conduit having an inlet end in fluid communication with the nozzle (90), and an outlet end adapted to deliver the biomaterial to the tissue site.

5. The device of claim 4, wherein the biomaterial transitions from the fluid state to a solid state after a set amount of time, a temperature change, or an exposure to an external stimuli such as radiation, UV light, or an electrical stimuli.

6. The device of claim 1, wherein the cartridge (80) is a dual-chambered cartridge for storing a first fluid biomaterial (82) in a first chamber (81) and a second fluid biomaterial (84) in a second chamber (83).

7. The device of claim 1, wherein the cartridge (80) further comprises a cartridge tip (86).

8. The device of claim 3, wherein the nozzle (90) further comprises a base (92) at a proximal end and a plurality of internal mixing fins.

9. The device of claim 8, wherein the distal end of the nozzle (90) is tapered.

10. The device of claim 4, wherein the tissue cavity access unit comprises an entry needle (52), an access cannula (54), and an obturator (53).

11. The device of claim 10, wherein the entry needle (52) has an outer diameter of about 0.010″ to about 0.100″ to gain initial access to the nucleus pulposus cavity.

12. The device of claim 10, wherein the cannula (54) has an outer diameter of about 0.050″ to about 0.400″ and the cannula (54) and the obturator (53) are adapted to dilate tissue of the annulus fibrosus (12).

13. The device of claim 12, wherein the cannula (54) and the obturator (53) are comprised of a thermopolymer such as PTFE, polyurethane, polyethylene, Pebax, polyester, polycarbonate, nylon, or delrin, or a metal such as stainless steel or Nitinol.

14. The device of claim 1, wherein the cartridge (80) mixes the biomaterial, which transitions from the fluid state to a solid state after approximately one minute.

15. The device of claim 1, wherein the cartridge (80) mixes the biomaterial, which transitions from the fluid state to a solid state after approximately three minutes.

16. The device of claim 1, wherein the cartridge (80) mixes the biomaterial, which transitions from the fluid state to a solid state after approximately five minutes.

17. The device of claim 1, wherein the biomaterial transitions from the fluid state to a solid state at a temperature between about 70° F. and about 120° F.

18. The device of claim 1, wherein the biomaterial transitions from the fluid state to the solid state at a temperature between about 85° F. and about 100° F.

19. The device of claim 6, wherein the first fluid biomaterial (82) includes hydrophilic poly(aldehyde) and the second fluid biomaterial (84) includes at least one of poly(amide), poly(amine) and poly(alcohol).

20. The device of claim 6, wherein the first fluid biomaterial (82) includes a poly(n-vinyl lactam) component and the second fluid biomaterial (84) includes a chitosan component.

21. The device of claim 1, wherein the biomaterial comprises a plurality of biomaterial components including a mixture of water and polyethyleneoxide/polypropyleneoxide (PEO-PPO) non-ionic block copolymer.

22. The device of claim 21, wherein the biomaterial components further comprise at least one of polyethyleneoxide (PEO) homopolymer, polypropyleneoxide (PPO) homopolymer, and other hydrophilic compounds including surfactants, alcohols, acids, salts, amines and mixtures thereof.

23. A method for producing an artificial nucleus pulposus implant in the nucleus cavity of the annulus fibrosus of a diseased disc to improve the natural anatomical and physiological function of the disc, comprising the steps of:
(a) obtaining access to the nucleus cavity;
(b) injecting the artificial nucleus pulposus (102) into the nucleus cavity, said artificial nucleus pulposus (102) comprising a phase changing biomaterial; and
(c) permitting the biomaterial to transition from a fluid state to a solid state in-situ after a given condition.

24. The method of claim 23, further comprising the step of removing the natural nucleus pulposus (14) from the nucleus cavity before the step of injecting the artificial nucleus pulposus (102) in the nucleus cavity.

25. The method of claim 23, wherein the phase changing biomaterial includes a plurality of biomaterial components.

26. The method of claim 23, wherein the biomaterial transitions from the fluid state to the solid state after a set amount of time, a temperature change, or an exposure to an external stimuli such as radiation, UV light, or an electrical stimuli.

27. The method of claim 24, wherein the natural nucleus pulposus (14) removing step includes one of irrigation, aspiration, chemonucleolysis, and grasping.

28. The method of claim 25, wherein the biomaterial components have a viscosity of less than about 5,000 cps in the fluid state and a viscosity of greater than about 100,000 cps in the solid state.

29. The method of claim 25, wherein the artificial nucleus pulposus injecting step further comprises the step of mixing the biomaterial components.

30. The method of claim 25, wherein the biomaterial components include a first fluid biomaterial (82) and a second fluid biomaterial (84).

31. The method of claim 23, wherein the biomaterial transitions from the fluid state to the solid state when exposed to UV light.

32. The method of claim 23, wherein the biomaterial transitions from the fluid state to the solid state when exposed to an electrical stimulation.

33. The method of claim 25, wherein the biomaterial components transition from the fluid state to the solid state approximately 1 minute after being mixed.

34. The method of claim 25, wherein the biomaterial components transition from the fluid state to the solid state approximately 3 minutes after being mixed.

35. The method of claim 25, wherein the biomaterial components transition from the fluid state to the solid state approximately 5 minutes after being mixed.

36. The method of claim 23, wherein the biomaterial transitions from the fluid state to the solid state at a temperature between about 70° F. and about 120° F.

37. The method of claim 23, wherein the biomaterial transitions from the fluid state to the solid state at a temperature between about 85° F. and about 100° F.

38. The method of claim 30, wherein the first fluid biomaterial (82) includes hydrophilic poly(aldehyde) and the second fluid biomaterial (84) includes at least one of poly(amide), poly(amine) and poly(alcohol).

39. The method of claim 30, wherein the first fluid biomaterial (82) includes a poly (n-vinyl lactam) component and the second fluid biomaterial (84) includes a chitosan component.

40. The method of claim 25, wherein the plurality of biomaterial components include a mixture of water and polyethyleneoxide/polypropyleneoxide (PEO-PPO) non-ionic block copolymer.

41. The method of claim 40, wherein the biomaterial components further comprise at least one of polyethyleneoxide (PEO) homopolymer, polypropyleneoxide (PPO) homopolymer, and other hydrophilic compounds including surfactants, alcohols, acids, salts, amines and mixtures thereof.

42. The method of claim 23, wherein the method is performed using endoscopic surgical instrumentation.

43. The method of claim 23, wherein the method is performed with the assistance of fluoroscopy or other imaging or resolution enhancing instrument.

44. A method for producing an artificial nucleus pulposus implant in the nucleus cavity of the annulus fibrosus of a diseased disc to improve the natural anatomical and physiological function of the disc, comprising the steps of:
(a) obtaining access to the nucleus cavity;
(b) inserting a scaffold in the nucleus cavity; and
(c) injecting the artificial nucleus pulposus (102) in the nucleus cavity, said artificial nucleus pulposus (102) comprising a phase changing biomaterial.

45. The method of claim 44, further comprising the step of permitting the biomaterial to transition from a fluid state to a solid state in-situ after a given condition.

46. The method of claim 44, further comprising the step of removing the natural nucleus pulposus (14) from the nucleus cavity before the step of injecting the artificial nucleus pulposus (102) in the nucleus cavity.

47. The method of claim 44, wherein the phase changing biomaterial includes a plurality of biomaterial components.

48. The method of claim 44, wherein the scaffold is made from preformed, extruded metal.

49. The method of claim 44, wherein the scaffold is made from preformed, extruded high durometer plastic such as polyurethane, polyethylene, silicone and PTFE.

50. The method of claim 44, wherein the scaffold is made of an injectable foam that solidifies in-situ.

51. A method for repairing a diseased disc to restore the natural anatomical and physiological function of the disc, comprising the steps of:
(a) providing an apparatus for delivering a phase changing biomaterial to the disc in a minimally invasive manner;
(b) providing said phase changing biomaterial to be injected to the disc; and
(c) permitting the biomaterial to transition from a fluid state to a solid state in situ after a given condition.

52. The method of claim 51, wherein the phase changing biomaterial includes a plurality of biomaterial components adapted to be mixed at the time of use to initiate cure.

53. The method of claim 52, further comprising the step of mixing the biomaterial components to initiate cure and delivering the mixed biomaterial to the disc in the fluid state.

54. The method of claim 51, further comprising the step of using minimally invasive techniques to remove damaged or diseased nucleus pulposus (14) from the disc.

55. The method of claim 51, wherein the apparatus for delivering said phase changing biomaterial to the disc comprises:
(a) a dispenser (70) comprising:
(i) a plunger (76) having a proximal portion and a distal portion, an inlet end and an outlet end,
(ii) a dispensing actuator (74) attached to the proximal portion of the plunger (76), and
(iii) a cartridge (80) adapted to be inserted into the inlet end of the plunger (76) for containing the phase changing biomaterial in a fluid state.

56. The method of claim 54, wherein the step of using minimally invasive techniques to remove the nucleus pulposus (14) from the disc includes at least one of irrigation, aspiration, chemonucleolysis, and grasping.
